(19) United States
(12) Patent Application Publication
(10) Pub. No.: US 2017/0311995 A1
(43) Pub. Date: Nov. 2, 2017

(54) SPINAL CORRECTION SYSTEM AND METHOD

(71) Applicant: Warsaw Orthopedic, Inc., Warsaw, IN (US)

(72) Inventor: Daniel Paxton Wall, Cordova, TN (US)

(21) Appl. No.: 15/141,931

(22) Filed: Apr. 29, 2016

(51) Int. Cl. A61B 17/70; A61B 17/02

(52) U.S. Cl. CPC A61B 17/7076; A61B 17/7079; A61B 17/025; A61B 17/708; A61B 17/7001; A61B 2017/0256

(57) ABSTRACT

A surgical instrument comprises a fulcrum including a first surface that defines a cavity configured for disposal of a first implant support such that the fulcrum is movable relative to the support. The first surface is rotatable relative to the fulcrum to engage the support and fix the first surface with the support. A second surface is engageable with a second implant support. In some embodiments, spinal constructs, implants, systems and methods are disclosed.

### TECHNICAL FIELD

The present disclosure generally relates to medical devices for the treatment of spinal disorders, and more particularly to a surgical system and method for correction of a spinal disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, corpectomy, discectomy, laminectomy, fusion, fixation and implantable prosthetics. Correction treatments used for positioning and alignment of vertebrae may employ implants, such as, for example, spinal constructs and interbody devices, for stabilization of a treated section of a spine. In some cases, the spinal constructs may be manipulated with surgical instruments for compression and distraction of vertebrae. This disclosure describes an improvement over these prior technologies.

### SUMMARY

In one embodiment, a surgical instrument is provided. The surgical instrument comprises a fulcrum including a first surface that defines a cavity configured for disposal of a first implant support such that the fulcrum is movable relative to the support. The first surface is rotatable relative to the fulcrum to engage the support and fix the first surface with the support. A second surface is engageable with a second implant support. In some embodiments, spinal constructs, implants, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a perspective view of the components shown in FIG. 1;

FIG. 3 is a perspective view in part phantom of the components shown in FIG. 1;

FIG. 4 is a cross section view of the components shown in FIG.

FIG. 5 is a perspective view of the components shown in FIG. 1;

FIG. 6 is a cross section view of the components shown in FIG.

FIG. 7 is a perspective view in part phantom of the components shown in FIG. 1;

FIG. 8 is a perspective view of components of one embodiment of a surgical system in accordance with the principles of the present disclosure disposed with vertebrae;

FIG. 9 is a perspective view of components of one embodiment of a surgical system in accordance with the principles of the present disclosure disposed with vertebrae;

FIG. 10 is a perspective view of components of one embodiment of a surgical system in accordance with the principles of the present disclosure;

FIG. 11 is a perspective view of the components shown in FIG. 10 with parts separated;

FIG. 12 is a cross section view of the components shown in FIG. 10; and

FIG. 13 is a cutaway view of the components shown in FIG. 10.

### DETAILED DESCRIPTION

The exemplary embodiments of the system and related methods of use disclosed are discussed in terms of medical devices for the treatment of musculoskeletal disorders and more particularly, in terms of a surgical system and method for correction of a spine disorder. In some embodiments, the present surgical system comprises one or more surgical instruments that can be employed with a spinal construct for treating a spine disorder. In some embodiments, the present surgical system can be employed with a pedicle subtraction osteotomy and a posterior vertebral column resection to correct angular and fixed kyphotic deformity, such as post traumatic deformity, congenital deformity and/or post infectious deformity.

In some embodiments, the present surgical system comprises a surgical instrument, such as, for example, an adjustable fulcrum compressor. In some embodiments, the surgical instrument includes a fulcrum style compressor that utilizes a fulcrum and a separate set of compression pliers. In some embodiments, the surgical instrument includes a fulcrum and pliers that are attached to screw extenders and actuated to either compress or distract vertebrae. In some embodiments, the surgical instrument includes a fulcrum that can be placed at multiple locations on screw extenders. In some embodiments, the present surgical system is employed with a method such that the pliers are drawn together, compressed and/or squeezed above the fulcrum and the disc space is distracted. In some embodiments, the present surgical system is employed with a method such that the pliers are drawn together, compressed and/or squeezed below the fulcrum and the disc space is compressed.

In some embodiments, the present surgical system comprises a surgical instrument including a fulcrum having a caulk gun style adjustment mechanism to allow the fulcrum to slide up and down the screw extenders. In some embodiments, the present surgical system is employed with a method such that the fulcrum includes a release button that is depressed so that a width of the fulcrum can be adjusted. In some embodiments, the present surgical system is employed with a method for compression and distraction of pedicle screws.

In some embodiments, the present surgical system is employed with a method that includes the step of actuating the fulcrum to allow the fulcrum to slide up and down an extender sleeve and to adjust the width of the fulcrum. In some embodiments, the method includes the step of releasing the fulcrum to lock the fulcrum in a selected position with the extenders. In some embodiments, the method includes the step of squeezing pliers below a selected position of the fulcrum to compress vertebrae. In some embodiments, the method includes the step of squeezing pliers above a selected position of the fulcrum to distract vertebrae.

In some embodiments, the present surgical system comprises a surgical instrument including a fulcrum having a hingedly connected actuator and stops to define a range of movement of the actuator relative to the fulcrum. In some embodiments, the actuator is connected to the fulcrum via a living hinge. In some embodiments, the fulcrum includes a retention plate fixed with the fulcrum with a screw. In some embodiments, the retention plate holds lock tabs and a spring in place for disposal of a slide of the fulcrum in a locking orientation. In some embodiments, the lock tabs move down and disengage a fulcrum rack when an actuator, such as, for example, a lever is fully depressed. In some embodiments, the fulcrum includes a slide having a shape that allows the fulcrum to be pulled or pushed axially relative to the fulcrum to adjust position of the slide for engaging an extender. In some embodiments, the slide includes a toothed rack engageable with lock tabs of the fulcrum.

In some embodiments, the lever is resiliently biased to lock the lever with a first extender. In some embodiments, the lever engages a first spring that allows the lever to collapse such that the fulcrum can slide up and down the first extender. In some embodiments, the lever engages a second spring to release a lock connected with a slide of the fulcrum. In some embodiments, the first spring comprises a first stage for releasing the first extender from a locking orientation with the fulcrum and the second spring comprises a second stage for releasing the slide and allowing the slide to axially translate relative to the fulcrum. In some embodiments, the lever includes a living hinge for releasing the first extender from a locking orientation with the fulcrum and the lever engages a release button having a spring for releasing the slide and allowing the slide to axially translate relative to the fulcrum.

In some embodiments, the present surgical system is employed with a method that can compress or distract and restore curvature of a spine. In some embodiments, the present surgical system includes instruments and tools for correcting a sagittal deformity and rebalancing a spine of a body. In some embodiments, the present surgical system is employed to treat degenerative deformities of a spine in a sagittal plane, for example, ankylosing spondylitis. In some embodiments, the present surgical system is employed to treat hyper-kyphosis, flat lumbar back and cervical hyper lordosis, including disorders that create an unbalance of a body and loss of alignment between body parts. In some embodiments, the present surgical system provides a selected amount of correction to apply a selected balance to a spine and provides control and adjustment to the amount of correction. In some embodiments, the present surgical system includes a series of tools and instruments that allow formulation of a type of correction applied and can control the correction stabilization using posterior instrumentation.

In some embodiments, one or all of the components of the surgical system are disposable, peel-pack, pre-packed sterile devices used with an implant. One or all of the components of the surgical system may be reusable. The surgical system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed surgical system and methods may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims. “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

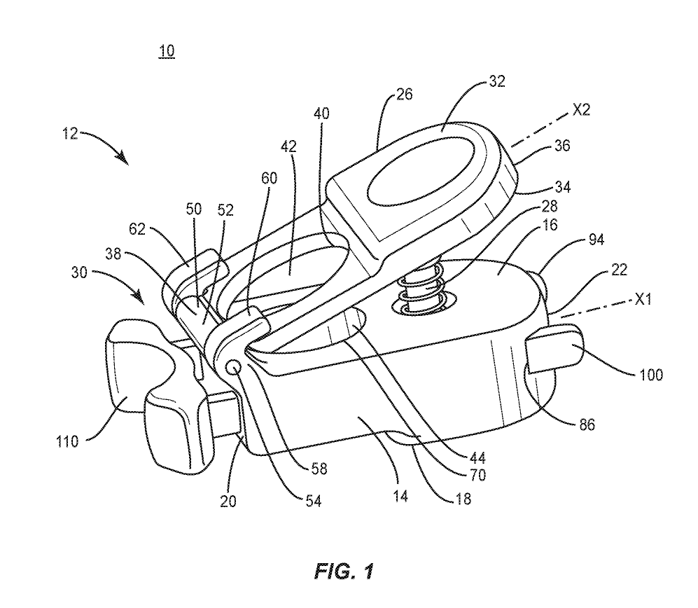

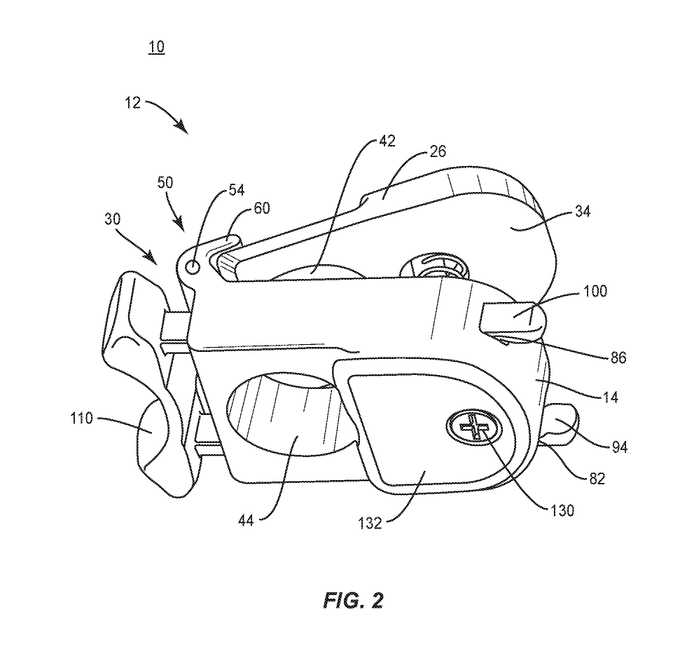

The following discussion includes a description of a surgical system and related methods of employing the surgical system in accordance with the principles of the present disclosure. Alternate embodiments are also disclosed. Reference is made to the exemplary embodiments of the present disclosure, which are illustrated in the accompanying figures. Turning to FIGS. 1-7, there are illustrated components of a surgical system, such as, for example, a spinal correction system 10.

The components of spinal correction system 10 can be fabricated from biologically acceptable materials suitable for medical applications, including metals, synthetic polymers, ceramics and bone material and/or their composites. For example, the components of spinal correction system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of spinal correction system 10 may have material composites, including the above materials, to achieve various desired characteristics such as strength, rigidity, elasticity, compliance, biomechanical performance, durability and radiolucency or imaging preference. The components of spinal correction system 10, individually or collectively, may also be fabricated from a heterogeneous material such as a combination of two or more of the above-described materials. The components of spinal correction system 10 may be monolithically formed, integrally connected or include fastening elements and/or instruments, as described herein.

Spinal correction system 10 is employed, for example, with a minimally invasive procedure, including percutaneous techniques, mini-open and open surgical techniques to deliver and introduce instrumentation and/or components of spinal constructs at a surgical site within a body of a patient, for example, a section of a spine. In some embodiments, one or more of the components of spinal correction system 10 are configured for engagement with instrumentation and/or spinal constructs attached with vertebrae to manipulate tissue and/or correct a spinal disorder, such as, for example, a sagittal deformity, as described herein. In some embodiments, spinal correction system 10 may be employed with surgical procedures, such as, for example, corpectomy, discectomy and/or fracture/trauma treatment and may include fusion and/or fixation that employ implants to restore the mechanical support function of vertebrae.

Spinal correction system 10 includes a surgical instrument, such as, for example a fulcrum 12. Fulcrum 12 includes a body 14 that defines an axis X1. Body 14 extends between a surface 16 and a surface 18. Body 14 extends between an end 20 and an end 22. In some embodiments, body 14 may extend in alternate configurations, such as, for example, arcuate, offset, staggered and/or angled portions, which may include acute, perpendicular and obtuse.

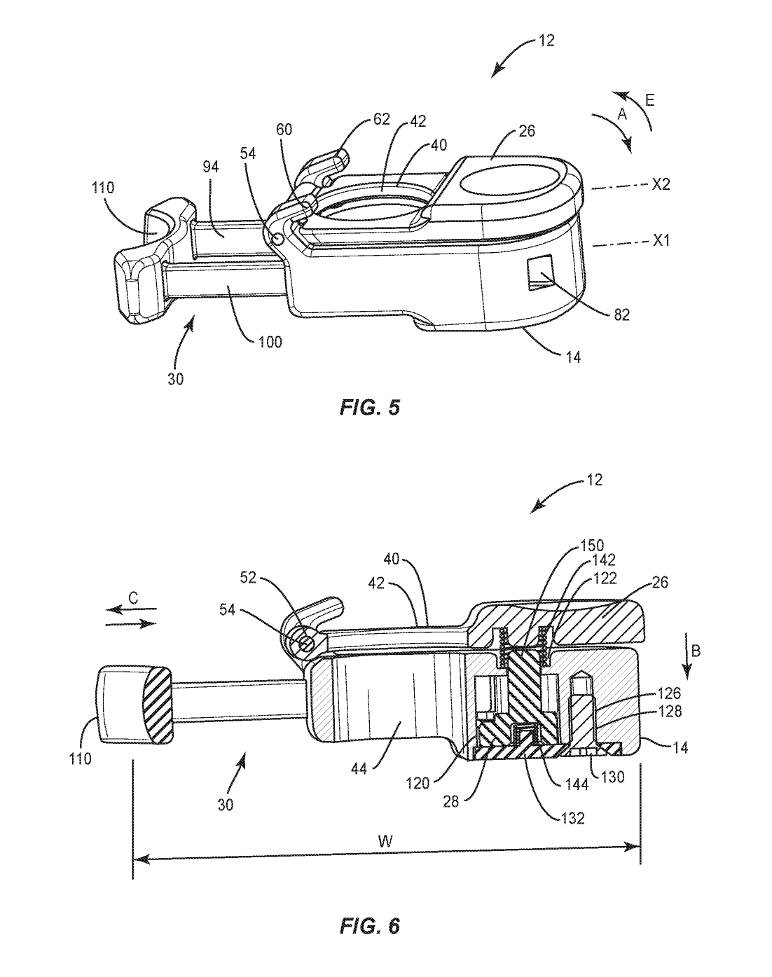

Body 14 includes a hinged connection with an actuator, such as, for example, a lever 26. Lever 26 is rotatable and/or pivotable relative to body 14 between a non-locking orientation, as shown in FIG. 5, and a locking orientation, as shown in FIG. 1, such that lever 26 engages an implant support, such as, for example, an extender 200, as described herein. In some embodiments, lever 26 is biased to the locking orientation, as described herein.

Lever 26 defines an axis X2. Lever 26 extends between a surface 32 and a surface 34. Lever 26 extends between an end 36 and an end 38. Lever 26 includes a surface 40 that defines a cavity, such as, for example, an opening 42. Opening 42 extends between surfaces 32, 24. In some embodiments, opening 42 includes a circular configuration.

Body 14 includes a surface 70 that defines an opening 44. Opening 44 extends between surfaces 16, 18. Opening 44 includes a circular configuration. In some embodiments, the cross-section geometry of opening 44 may have various configurations, such as, for example, round, oval, oblong, triangular, polygonal having planar or arcuate side portions, irregular, uniform, non-uniform, consistent or variable.

Opening 42 is configured for alignment with an opening 44 disposed with body 14 to facilitate translation of fulcrum 12 in a non-locking orientation of lever 26 relative to an implant support, as described herein. In some embodiments, in a locking orientation of lever 26, surface 40 supports an implant support and/or may engage the implant support disposed with opening 44 in a friction fit engagement such that surface 40 resists and/or prevents translation of the implant support relative to fulcrum 12. In some embodiments, in the locking orientation of lever 26, surface 40 is rotated such that opening 42 is offset and/or alternately aligned with opening 44.

In some embodiments, the cross-section geometry of opening 42 may have various configurations to facilitate connection with an implant support, as described herein, such as, for example, round, oval, oblong, triangular, polygonal having planar or arcuate side portions, irregular, uniform, non-uniform, consistent or variable. In some embodiments, lever 26 may extend in alternate configurations, such as, for example, arcuate, offset, staggered and/or angled portions, which may include acute, perpendicular and obtuse.

Surface 32 includes a recess 46 configured to facilitate gripping of lever 26 to rotate lever 26 relative to body 14 about a hinge 50, as described herein. Lever 26 is pivotable relative to body 14 between a locking orientation and a non-locking orientation with an implant support, as described herein.

Lever 26 includes hinge 50 connected with body 14. Hinge 50 includes a tube 52 and a pin 54. Tube 52 extends along end 38. Tube 52 includes a cavity configured for disposal of pin 54. In some embodiments, hinge 50 may include, such as, for example, a post, screw, resilient hinge, ratchet and/or concentric parts. Hinge 50 facilitates rotation of lever 26 in a range of movement relative to body 14 between a non-locking orientation, as shown in FIG. 5, and a locking orientation, as shown in FIG. 1, as described herein. In some embodiments, in a locking orientation, axis X2 is disposed transverse to axis X1 and surface 40 is disposed in a friction fit engagement with an implant support. In some embodiments, in a non-locking orientation, axis X2 is disposed parallel to axis X1 such that fulcrum 12 is translatable relative to an implant support.

Body 14 includes a surface 58 that defines a stop 60 and a stop 62. Stops 60, 62 are connected with pin 54 to facilitate rotation of lever 26 in a range of movement relative to body 14. Stop 60 includes a surface 64. Stop 62 includes a surface 66. Surfaces 64, 66 are engageable with lever 26 to limit rotation of lever 26 at a locking orientation position, as shown in FIG. 1. In some embodiments, surfaces 64, 66 are configured to define a range of motion of lever 26 relative to body 14 between a non-locking position, as shown in FIG. 5, and a locking position.

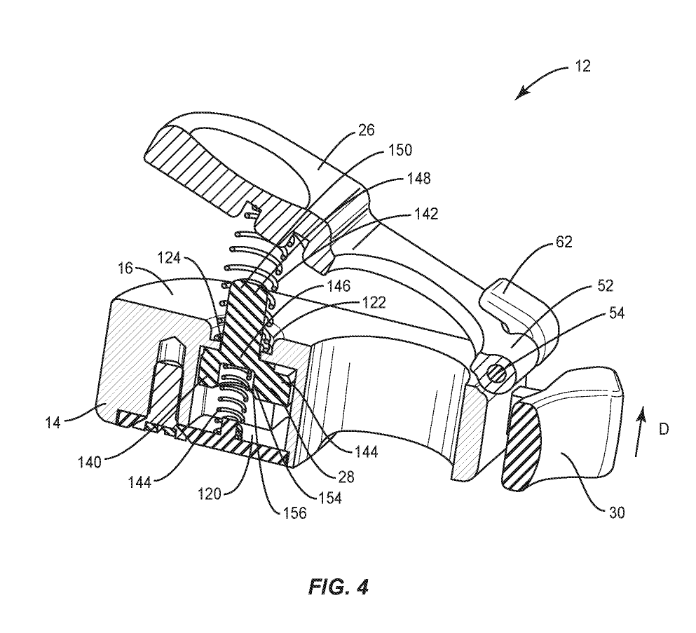

Body 14 is configured for disposal of a lock 28. Lock 28 is actuated by lever 26 between a non-locking orientation of lock 28 and slide 30, as shown in FIG. 6, to facilitate translation of slide 30 relative to body 14 into and out of engagement with an implant support, such as, for example, an extender 202, and a locking orientation of lock 28 and slide 30, as shown in FIG. 4, to fix position of slide 30 relative to body 14, as described herein. For example, in a locking orientation of lock 28 and slide 30, a surface of slide 30 can engage extender 202 to fix a position of body 14 therewith.

Body 14 includes a surface 80 that defines a passageway 82. Passageway 82 extends between ends 20, 22 of body 14. Passageway 82 extends parallel to axis X1. Passageway 82 is configured for disposal of a leg of slide 30, as described herein. Body 14 includes a surface 84 that defines a passageway 86. Passageway 86 extends between ends of body 14. Passageway 86 extends parallel to axis X1. Passageway 86 is configured for disposal of a leg of slide 30, as described herein.

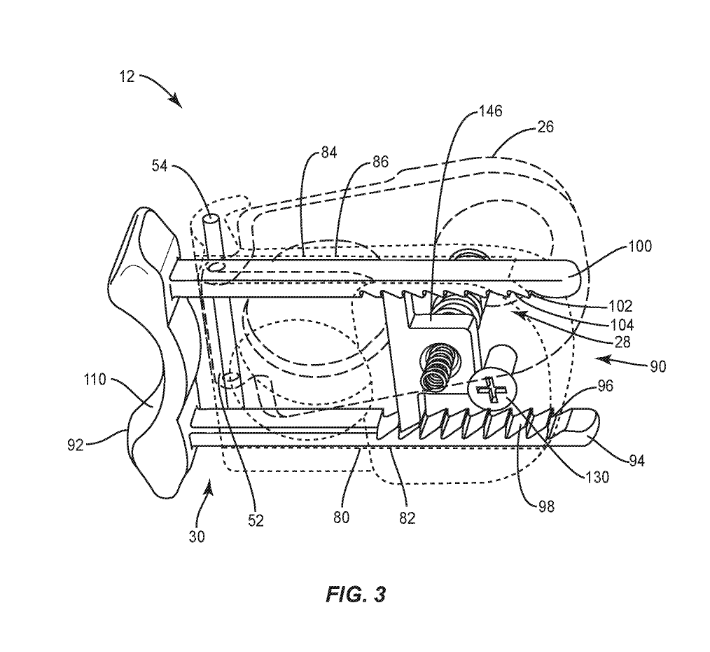

Slide 30 extends between an end 90 and an end 92, as shown in FIG. 3. Slide 30 includes a leg 94 extending between ends 90, 92. Leg 94 extends parallel to axis X1 Leg 94 includes a surface 96 that includes a plurality of teeth defining a toothed rack 98. Rack 98 is engageable with lock 28 in a locking orientation of lock 28 and slide 30 to resist and/or prevent translation of slide 30 relative to body 14. Rack 98 is moveable out of engagement with lock 28 to facilitate selective translation of slide 30 relative to body 14 in a non-locking orientation of lock 28 and slide 30, as described herein.

Slide 30 includes a leg 100 extending between ends 90, 92. Leg 100 extends parallel to axis X1. Leg 100 includes a surface 102 that includes a plurality of teeth defining a toothed rack 104. Rack 104 is engageable with lock 28 in a locking orientation of lock 28 and slide 30 to resist and/or prevent translation of slide 30 relative to body 14. Rack 104 is moveable out of engagement with lock 28 to facilitate selective translation of slide 30 relative to body 14 in a non-locking orientation of lock 28 and slide 30, as described herein. Lock 28 is disposable into and out of engagement with slide 30 to facilitate translation of slide 30 relative to body 14 along axis X1 to increase and/or decrease a width w of fulcrum 12 to facilitate engagement with an implant support.

End 92 includes a surface 110. Surface 110 is configured for engagement with an implant support. In some embodiments, surface 110 includes a concave configuration to facilitate engagement with a surface of an implant support to fix position of fulcrum 12 with the implant support. Slide 30 is translatable relative to body 14 within passageways 82, 86 to orient surface 110 with an implant support. In a non-locking orientation of lever 26, lock 28 is disengaged from racks 98, 104 allowing slide 30 to selectively translate within passageways 82, 86 relative to body 14. Surface 110 is moveable relative to body 14 for positioning with an implant support. In a locking orientation, slide 30 is fixed relative to body 14 such that lock 28 is engaged with racks 98, 104 to fix surface 110 with an implant support. In some embodiments, surface 110 is engaged with the surface of extender 202 in a friction fit engagement.

Lock 28 is configured for disposal within a channel 120 of body 14. Channel 120 is configured to facilitate translation of lock 28 between a locking orientation and a non-locking orientation relative to slide 30, as described herein. In some embodiments, the cross-section geometry of channel 120 may have various configurations, such as, for example, round, oval, oblong, triangular, polygonal having planar or arcuate side portions, irregular, uniform, non-uniform, consistent, variable, horseshoe shape, U-shape or kidney bean shape.

Surface 16 defines a recess 122. Recess 122 includes an opening 124. Opening 124 is in communication with channel 120 to receive a portion of lock 28, as described herein. Body 14 includes a surface 126 that defines a cavity 128. Surface 126 is configured for engagement of a coupling member, such as, for example, a screw 130. Screw 130 is configured to connect a plate 132 with body 14 to retain lock 28 within body 14.

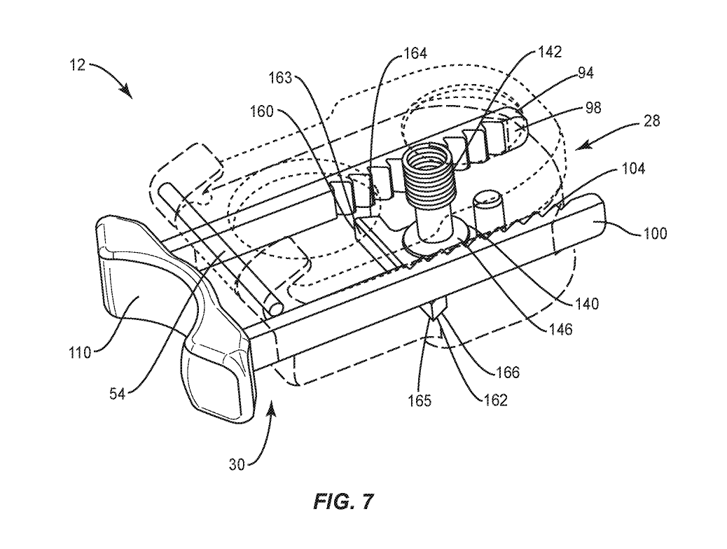

Lock 28 includes a member 140 that is spring loaded to a locking orientation by a two-stage spring system including springs 142, 144. The two stage spring system biases lever 26 and lock 28 and slide 30 in a locking orientation, as described herein. Member 140 includes a base 146. Base 146 includes a surface 148 that defines a button 150. Button 150 is oriented perpendicular to axis X1 within channel 120. Button 150 extends through opening 124. Button 150 includes a surface 152 engageable with surface 34. Button 150 is configured for disposal with spring 142, as shown in FIGS. 4 and 7. Spring 142 is configured to bias lever 26 in a locking orientation.

Base 146 includes a surface 154 that defines a cavity 156. Cavity 156 is configured for disposal of spring 144. Spring 144 is configured to bias lock 28 in a locking orientation with slide 30. Actuation of lever 26 causes lever 26 to overcome the force applied by spring 142. Engagement of button 150 is configured to overcome the force applied by spring 144 to disengage lock 28 from slide 30, as described herein.

Member 140 includes a tab 160 and a tab 162, as shown in FIG. 7. Tab 160 includes a planar surface 163 configured to engage planar mating surfaces of rack 98 to fix position of slide 30 with body 14 and an angled surface 164 that slides along angled mating surfaces of rack 98. Tab 162 includes a planar surface 165 configured to engage planar mating surfaces of rack 104 to fix position of slide 30 with body 14 and an angled surface 166 that slides along angled mating surfaces of rack 104. In the locking orientation of lock 28 and slide 30, tabs 160, 162 slide along the angled mating surfaces of racks 98, 104 in an axial direction to adjust slide 30 relative to body 14 such that surface 110 is translated into engagement an implant support. In the locking orientation of lock 28 and slide 30, surfaces 163, 165 engage the planar mating surfaces of racks 98, 104 in an opposing axial direction to selectively fix slide 30 relative to body 14, for example, upon engagement of surface 110 with the implant support. In the non-locking orientation of lock 28 and slide 30, member 140 is disengaged from slide 30, as described herein.

Lever 26 is actuated to translate lock 28 between a locking orientation, as shown in FIG. 4, and a non-locking orientation, as shown in FIG. 6, to facilitate translation of slide 30. Lever 26 is rotated, in a direction shown by arrow A in FIG. 5, into a non-locking orientation relative, for example, to extender 200 such that lever 26 engages button 150. Engagement of button 150 causes lock 28 to translate, in a direction shown by arrow B in FIG. 6, to disengage from legs 94, 100 from slide 30 into a non-locking orientation of lock 28 and slide 30. Translation of lock 28 causes surfaces 164, 166 to disengage from racks 98, 104. In some embodiments, lock 28 and slide 30 are disposed in a non-locking orientation to translate legs 94, 100 in opposing axial directions within passageways 82, 86 for positioning surface 110 with extender 202. In some embodiments, lock 28 and slide 30 are disposed in a locking orientation, such that legs 94, 100 slide along member 140, as described herein, in an axial direction to adjust slide 30 so that surface 110 engages extender 202. Upon selective disposal of slide 30 in position with extender 202, in the locking orientation of lock 28 and slide 30, translation of slide 30 relative to body 14 in an opposing axial direction is resisted and/or prevented to selectively fix surface 110 with extender 202. In some embodiments, slide 30 is translated in the directions shown by arrows C in FIG. 6, to selectively adjust width w of fulcrum 12 to facilitate engagement with extender 202.

Lever 26 is releasable and springs 142, 144 bias lock 28, in a direction shown by arrow D in FIG. 4, causing surfaces 164, 166 to translate into engagement with racks 98, 104 into a locking orientation of slide 30 and lock 28. In the locking orientation, racks 98, 104 resist and/or prevent translation of slide 30 thereby fixing slide 30 relative to body 14. In some embodiments, surface 110 is disposed in a friction fit engagement with extender 202 in a locking orientation of lever 26.

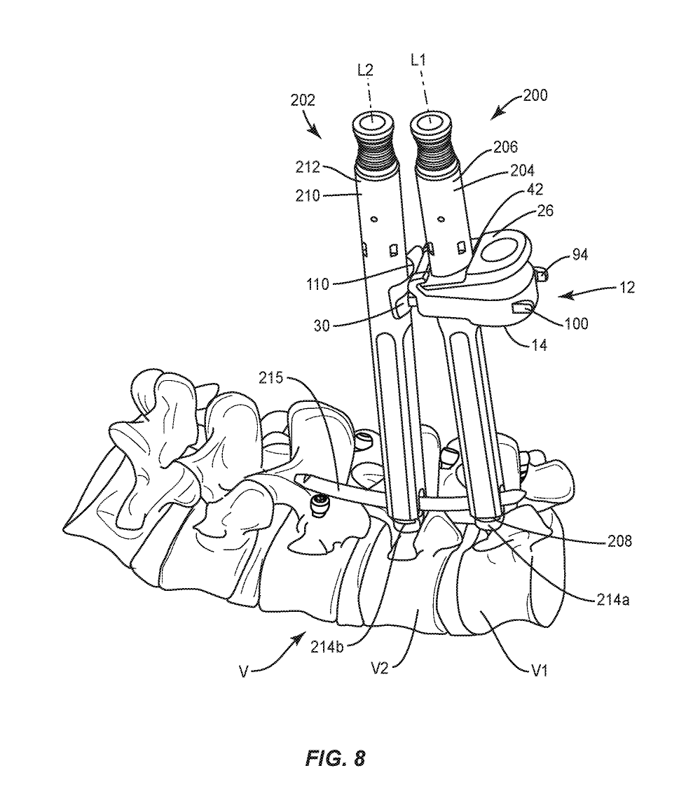

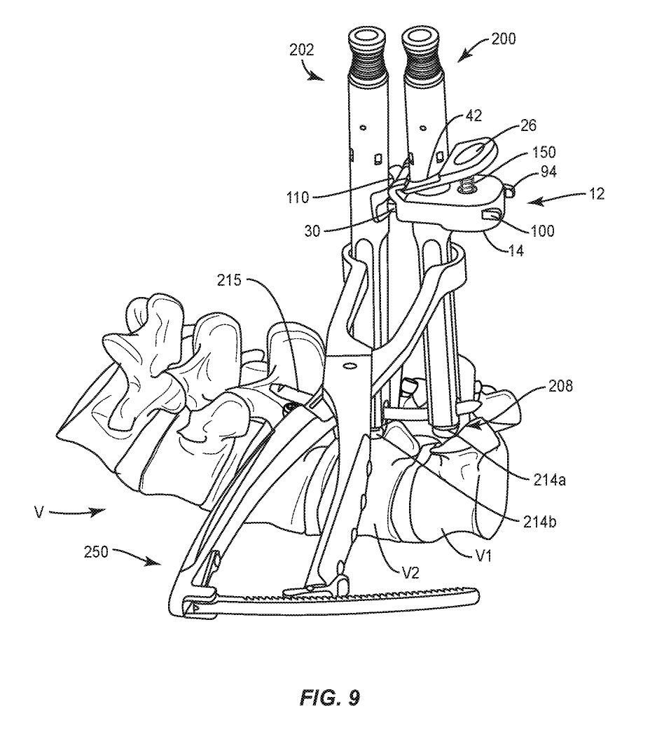

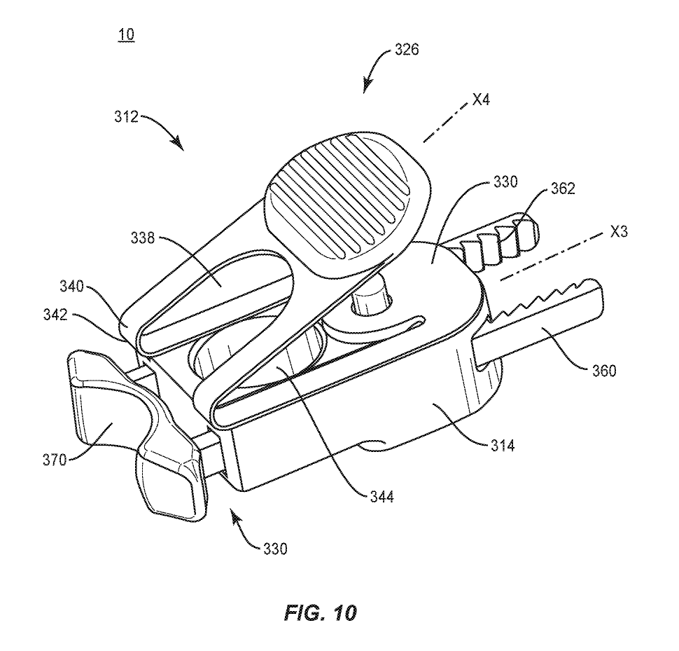

Extender 200 includes a member, such as, for example, a sleeve 204. In some embodiments, sleeve 204 is configured as a reinforcement sleeve for connection with one or more spinal implants and/or constructs, surgical instruments and/or implant supports. Sleeve 204 extends along a longitudinal axis L1 and includes a surface 206. Surface 206 is configured for engagement with surface 80 of fulcrum 12 to facilitate manipulation of extender 200, as described herein. Sleeve 204 is configured for translation over a portion of a spinal construct 208, as shown in FIGS. 8 and 9. Sleeve 204 provides a counter-torque sleeve engageable with spinal construct 208 to resist and/or prevent relative movement therebetween. In some embodiments, sleeve 204 provides a counter-torque sleeve engageable with spinal construct 208 for treating spinal trauma and/or deformity disorders with a minimally invasive surgical technique. In some embodiments, sleeve 204 provides a counter-torque sleeve engageable with spinal construct 208 for employment with a trauma rack and/or small deformity maneuvers, as described herein. In some embodiments, sleeve 204 provides a counter-torque sleeve engageable with spinal construct 208 for manipulation of vertebrae during a surgical treatment, for example, to displace, pull, twist or align vertebrae.

Extender 202, similar to extender 200, includes a member, such as, for example, a sleeve 210. Sleeve 210 extends along a longitudinal axis L2 and includes a surface 212. Surface 212 is configured for engagement with surface 180 of fulcrum 12 to facilitate manipulation of extender 202, as described herein. Sleeve 210 is configured for translation over a portion of spinal construct 208, as shown in FIGS. 8 and 9, similar to extender 200.

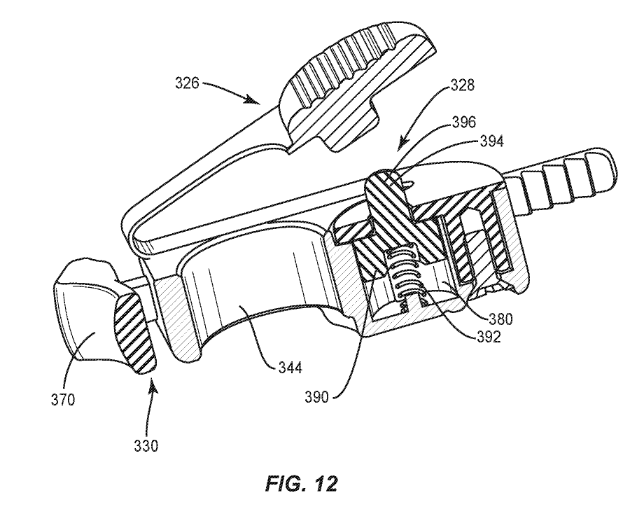

In assembly, operation and use, spinal correction system 10 including fulcrum 12, similar to the systems and methods described with regard to FIGS. 1-7 and 10-13, is employed with a surgical procedure for treatment of a spine of a patient including vertebrae V, as shown in FIGS. 8 and 9. For example, spinal correction system 10 can be employed with a surgical correction treatment of an applicable condition or injury of an affected section of a spinal column and adjacent areas within a body, such as, for example, vertebral levels V1, V2 of vertebrae V. In some embodiments, spinal correction system 10 may be employed with one or a plurality of vertebra.

In use, to treat the affected section of vertebrae V, a medical practitioner obtains access to a surgical site including vertebrae V in any appropriate manner, such as through incision and retraction of tissues. In some embodiments, spinal correction system 10 may be used in any existing surgical method or technique including open surgery, mini-open surgery, minimally invasive surgery and percutaneous surgical implantation, whereby vertebrae V is accessed through a mini-incision, or sleeve that provides a protected passageway to the area. An incision is made in the body of the patient and a cutting instrument (not shown) creates a surgical pathway for implantation of components of spinal correction system 10. A preparation instrument (not shown) can be employed to prepare tissue surfaces of vertebrae V, as well as for aspiration and irrigation of a surgical region.

An incision is made in the body of a patient and a cutting instrument (not shown) creates a surgical pathway for implantation of components of spinal correction system 10. A preparation instrument (not shown) can be employed to prepare tissue surfaces of vertebrae V, as well as for aspiration and irrigation of a surgical region.

Pilot holes or the like are made in selected vertebra V1 and V2 for receiving bone fasteners 214a, 214b of spinal construct 208. A driver (not shown) is disposed adjacent vertebrae V at a surgical site and is manipulated to drive, torque, insert or otherwise connect bone fasteners 214a, 214b with vertebrae V1 and V2. A spinal rod 215 is connected with bone fasteners 214a, 214b. Extenders 200, 202 are delivered to the surgical site adjacent vertebrae V and oriented for manipulation, alignment and capture of spinal construct 208. For example, extender 200 is disposed with bone fastener 214a and an adjacent portion of spinal rod 215 and extender 202 is disposed with bone fastener 214b and an adjacent portion of spinal rod 215.

Fulcrum 12 is disposed adjacent extenders 200, 202. Lever 26 is initially disposed in a locking orientation, as described herein. Lever 26 is rotated into the non-locking orientation such that opening 42 is disposed in alignment with opening 44. Extender 200 is disposed within openings 42, 44 and translated through body 14 for selective orientation with fulcrum 12. Fulcrum 12 is movable along extender 200 for selective positioning therewith in connection with a surgical procedure, as described herein. In some embodiments, fulcrum 12 is disposed with extender 200 to provide a pivot point for extenders 200, 202 to facilitate compression and/or distraction of vertebrae V.

Lever 26 is releasable and rotates into a locking orientation, as described herein, such that surface 40 is engaged with surface 206 of extender 200 in a friction fit to dispose fulcrum 12 in a selected position and fixed orientation with extender 200. The inner surfaces of body 14 engage the outer surfaces of extender 200 to maintain the fixed orientation and to resist and/or prevent movement of fulcrum 12 relative to extender 200 in rotation and/or translation.

In the locking orientation of lock 28 and slide 30, slide 30 is adjustable in an axial direction relative to body 14, as described herein, such that surface 110 is translated into engagement with extender 202. In the locking orientation of lock 28 and slide 30, lock 28 engages slide 30 to resist and/or prevent translation of slide 30 in an opposing axial direction, as described herein, to selectively fix slide 30 relative to body 14 upon engagement of surface 110 with extender 202. Surface 110 is engaged with surface 212 to facilitate compression and/or distraction of vertebrae V. As such, fulcrum 12 is releasably fixed with extenders 200, 202 to facilitate compression and/or distraction of vertebrae V. In some embodiments, lever 26 is rotated to engage button 150, as described herein, and lock 28 and slide 30 are disposed in a non-locking orientation to translate slide 30 in opposing axial directions for adjustable positioning of surface 110 with extender 202.

A surgical instrument, such as, for example, pliers 250 are engaged with extenders 200, 202, as shown in FIG. 9. Pliers 250 are connected with extenders 200, 202, which are engaged with spinal construct 208, in a configuration to rotate vertebrae V1, V2 about fulcrum 12, for example, in a compression and/or distraction treatment of vertebrae V. Positioning of pliers 250 below fulcrum 12 compresses vertebrae V about fulcrum 12. Positioning of pliers 250 above fulcrum 12 distracts vertebrae V about fulcrum 12. In some embodiments, pliers 250 are engaged with extenders 200, 202 to rotate vertebrae V1, V2 about fulcrum 12 for manipulating vertebrae V during a surgical correction treatment to derotate, displace, pull, twist or align vertebrae V to a selected orientation for sagittal, coronal and/or axial correction.

In some embodiments, use of microsurgical and image guided technologies may be employed to access, view and repair spinal deterioration or damage, with the aid of spinal correction system 10. Upon completion of the procedure, the non-implanted components, surgical instruments and assemblies of spinal correction system 10 are removed and the incision is closed.

In some embodiments, spinal correction system 10 comprises a kit including a plurality of interbody devices, plates, bone fasteners and/or fixation elements, which may be employed with a single vertebral level or a plurality of vertebral levels. In some embodiments, the fasteners may be engaged with vertebrae in various orientations, such as, for example, series, parallel, offset, staggered and/or alternate vertebral levels. In some embodiments, the fasteners may be configured as multi-axial screws, sagittal angulation screws, pedicle screws, mono-axial screws, uni-planar screws, fixed screws, anchors, tissue penetrating screws, conventional screws and expanding screws. In some embodiments, the fasteners may be employed with wedges, anchors, buttons, clips, snaps, friction fittings, compressive fittings, expanding rivets, staples, nails, adhesives, posts, connectors, fixation plates and/or posts. In some embodiments, spinal correction system 10 includes surgical instruments, such as, for example, inserters, extenders, reducers, spreaders, distracters, blades, retractors, clamps, forceps, elevators and drills, which may be alternately sized and dimensioned, and arranged as a kit.

In some embodiments, spinal correction system 10 includes an agent, which may be disposed, packed, coated or layered within, on, adjacent or about the components and/or surfaces of spinal correction system 10, and/or disposed with tissue. In some embodiments, the agent may include bone growth promoting material, such as, for example, bone graft to enhance fixation of the components and/or surfaces of spinal correction system 10 with vertebrae. In some embodiments, the agent may include one or a plurality of therapeutic agents and/or pharmacological agents for release, including sustained release, to treat, for example, pain, inflammation and degeneration.

In one embodiment, as shown in FIGS. 10-13, spinal correction system 10, similar to the systems and methods described herein, includes a fulcrum 312, similar to fulcrum 12 as described herein.

Fulcrum 312 includes a body 314, similar to body 14 described herein, which defines an axis X3. Body 314 extends between a surface 316 and a surface 318. Body 314 extends between an end 320 and an end 322. Body 314 is configured for connection with a lever 326, similar to lever 26 described herein. Lever 326 is rotatable and/or pivotable relative to body 314 between a non-locking orientation and a locking orientation, similar to that described herein. In some embodiments, lever 326 is biased to the locking orientation, as described herein.

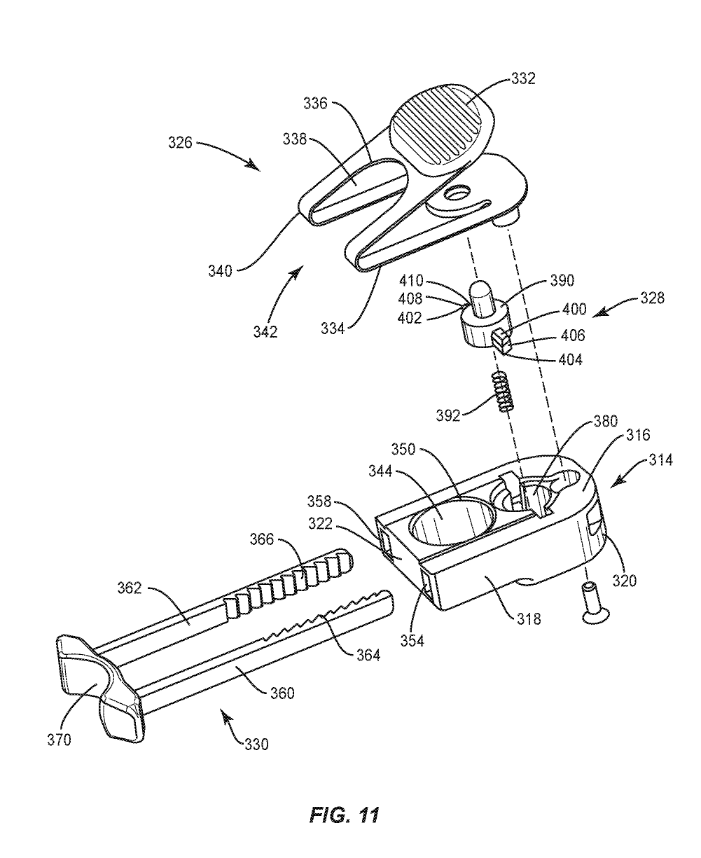

Lever 326 includes a portion 332 and a portion 334. Portion 332 includes a surface 336 that defines a cavity, such as, for example, an opening 338, as shown in FIG. 11. Portions 332, 334 are connected at an end 340. End 340 includes a living hinge 342 disposed between portions 332, 334 to facilitate rotation of portion 332 relative to portion 334 and body 314 to engage lock 328, as described herein. Hinge 342 is configured to bias portion 332 into a locking orientation. Hinge 342 is configured to facilitate rotation of portion 332 between a non-locking orientation and a locking orientation of lever 326 relative to an implant support, similar to that described herein. In some embodiments, lever 326 is manufactured in an injection molding process that creates portions 332, 334 and end 340 at one time as a single piece to form living hinge 342.

Portion 334 is connectable with body 314. Portion 334 includes a surface 346 that defines a cavity 348. Cavity 348 is configured for disposal of lock 328, as shown in FIG. 10 and similar to lock 28 described herein. In some embodiments, portion 334 may be attached with body 314 with, such as, for example, anchors, buttons, clips, snaps, friction fittings, compressive fittings, expanding rivets, staples, nails, adhesives, posts, connectors, fixation plates and/or posts.

Body 314 includes a surface 350 that defines opening 344. Opening 344 extends between surfaces 316, 318. Opening 338 is configured for alignment with opening 344 in a non-locking orientation of lever 326 relative to an implant support, as described herein. In some embodiments, in a locking orientation of lever 326, surface 336 supports an implant support and/or may engage the implant support disposed with opening 344 in a friction fit engagement such that surface 336 resists and/or prevents rotation and/or translation of the implant support relative to fulcrum 312. In some embodiments, in the locking orientation of lever 326, surface 336 is rotated such that opening 338 is offset and/or alternately aligned with opening 344.

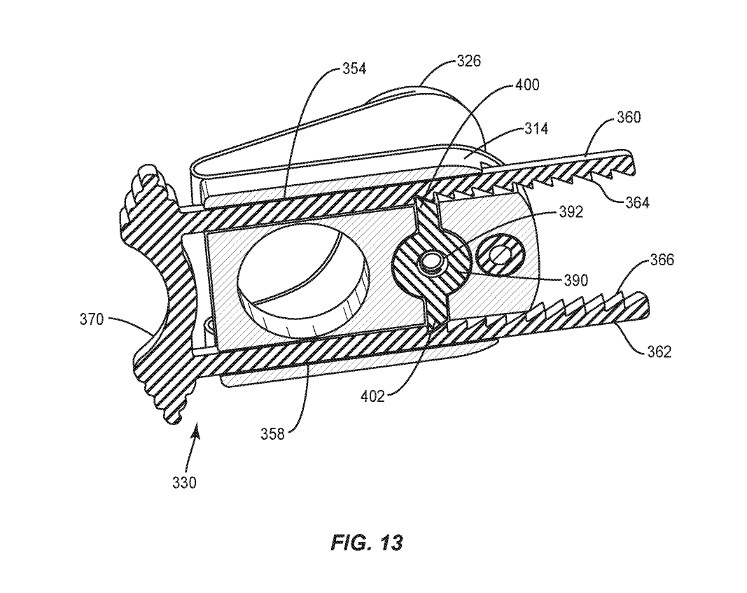

Body 314 includes a passageway 354 configured for disposal of a leg of slide 330, similar to slide 30 described herein. Body 314 includes a passageway 358 configured for disposal of a leg of slide 330. Slide 330 includes a leg 360 and a leg 362. Legs 360, 362 include racks 364, 366, as shown in FIG. 13. Lock 328 is configured for disengagement from racks 364, 366 in a non-locking orientation of lock 328 and slide 330 for selective translation of slide 330, as described herein. Slide 330 is configured for translation along axis X3 to increase and/or decrease a width of fulcrum 312 to facilitate engagement with an implant support, similar to that described herein. Racks 364, 366 are configured for engagement with lock 328 in a locking orientation of lock 328 and slide 330 to adjust slide 330 in a first axial direction and to fix slide 330 in an opposing axial direction relative to body 314 and an implant support, as described herein.

Slide 330 includes a surface 370 configured for engagement with an implant support, as described herein. Slide 330 is translatable within passageways 354, 358 to orient surface 370 with an implant support, as described herein. In a non-locking orientation of lock 328 and slide 330, lock 328 is disengaged from racks 364, 366 allowing slide 330 to selectively translate within passageways 354, 358 relative to body 314. In a locking orientation of lock 328 and slide 330, slide 330 is fixed relative to body 314 such that lock 328 is engaged with racks 364, 366 to fix surface 370 with an implant support, as described herein.

Lock 328 is configured for disposal within a channel 380 of body 314. Channel 380 is configured to facilitate translation of lock 328 between a locking orientation and a non-locking orientation of lock 328 and slide 330, as described herein. Lock 328 includes a member 390 that is spring loaded into the locking orientation by a spring 392. Member 390 includes a surface 394 that defines a button 396. Button 396 is engageable with lever 326 to apply a force to button 396 to overcome the spring force applied to lock 328.

Member 390 includes a tab 400 and a tab 402, as shown in FIGS. 11 and 13. Tab 400 includes a planar surface 404 configured to engage planar mating surfaces of rack 364 to fix position of slide 330 with body 314 and an angled surface 406 that slides along angled mating surfaces of rack 364. Tab 402 includes a planar surface 408 configured to engage planar mating surfaces of rack 366 to fix position of slide 330 with body 314 and an angled surface 410 that slides along angled mating surfaces of rack 366. In the locking orientation of lock 328 and slide 330, tabs 400, 402 slide along the angled mating surfaces of racks 364, 366 in an axial direction to adjust slide 330 relative to body 314 such that surface 370 is translated into engagement with an implant support, as described herein. In the locking orientation of lock 328 and slide 330, surfaces 404, 408 engage the planar mating surfaces of racks 364, 366 in an opposing axial direction to selectively fix slide 330 relative to body 314, for example, upon engagement of surface 370 with the implant support. Lever 326 is actuated to translate lock 328 between a locking orientation and a non-locking orientation to facilitate translation of slide 330, as described herein.

Lever 326 is releasable and spring 392 biases lock 328 causing surfaces 406, 410 to translate into engagement with racks 364, 366 into a locking orientation of slide 330 and lock 328. In the locking orientation, racks 364, 366 resist and/or prevent translation of slide 330 thereby fixing slide 330 relative to body 314. In some embodiments, surface 370 is disposed in a friction fit engagement with the implant support in a locking orientation of lever 326, as described herein.

In the locking orientation of lock 328 and slide 330, slide is adjustable in an axial direction relative to body 314, as described herein, such that surface 370 is translated into engagement with the implant support. In the locking orientation of lock 328 and slide 330, lock 328 engages slide 330 to resist and/or prevent translation of slide 330 in an opposing axial direction, as described herein, to selectively fix slide 330 relative to body 314 upon engagement of surface 370 with the implant support, as described herein. Surface 370 is engaged with a surface of the implant support to facilitate compression and/or distraction of vertebrae, as described herein. As such, fulcrum 312 is releasably fixed with the implant supports to facilitate compression and/or distraction of the vertebrae. In some embodiments, lever 326 is rotated to engage button 396, as described herein, and lock 328 and slide 330 are disposed in a non-locking orientation to translate slide 330 in opposing axial directions for adjustable positioning of surface 370 with the implant support.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a fulcrum including a first surface that defines a cavity configured for disposal of a first implant support such that the fulcrum is movable relative to the support, the first surface being rotatable relative to the fulcrum to engage the support and fix the first surface with the support, and
a second surface being engageable with a second implant support.

2. A surgical instrument as recited in claim 1, wherein the second surface is movable relative to the fulcrum to engage the second support.

3. A surgical instrument as recited in claim 1, wherein the second surface is disposable in a non-locking orientation such that the second surface is axially translatable relative to the fulcrum and a locking orientation to resist and/or prevent movement of the second surface relative to the fulcrum.

4. A surgical instrument as recited in claim 3, wherein the fulcrum includes a lock engageable with the second surface to fix the second surface in the locking orientation.

5. A surgical instrument as recited in claim 4, wherein the fulcrum includes a body and a slide including the second surface and a toothed rack engageable with the lock.

6. A surgical instrument as recited in claim 4, wherein the lock is resiliently biased to the locking orientation.

7. A surgical instrument as recited in claim 4, wherein the first surface is engageable with the lock to dispose the lock in the non-locking orientation.

8. A surgical instrument as recited in claim 1, wherein the first surface is movable relative to the fulcrum between a non-locking orientation such that the first support is movable relative to the first surface and a locking orientation to fix the first surface with the first support.

9. A surgical instrument as recited in claim 1, wherein the fulcrum includes a body that defines an opening aligned with the cavity for disposal of the first support.

10. A surgical instrument as recited in claim 1, wherein the fulcrum includes an actuator having the first surface and is connected to the fulcrum via a hinge.

11. A surgical instrument as recited in claim 10, wherein the fulcrum includes at least one stop engageable with the actuator.

12. A surgical instrument as recited in claim 10, wherein the fulcrum includes a first stop and a second stop, the stops being engageable with the actuator to define a range of rotation of the actuator relative to the fulcrum.

13. A surgical instrument as recited in claim 1, wherein the fulcrum includes an actuator having the first surface and is connected to the fulcrum via a living hinge.

14. A surgical instrument as recited in claim 1, wherein the first surface is fixed with the first support via a friction fit engagement.

15. A surgical instrument as recited in claim 1, wherein the second surface is fixed with the second support via a friction fit engagement.

16. A surgical instrument as recited in claim 1, wherein the second surface defines a concave cavity and a grip surface adjacent thereto.

17. A surgical instrument comprising:
a fulcrum defining an opening configured for disposal of a first implant support;
an actuator connected to the fulcrum via a hinge and defining an opening for disposal of the support, the actuator being movable relative to the fulcrum between a non-locking orientation such that the first support is movable to a selected position along a surface of the support and a locking orientation to fix the actuator with the first support at the selected position; and
a slide being engageable with a second implant support to fix the slide with the second support, the slide being disposable in a non-locking orientation such that the slide is axially translatable relative to the fulcrum and a locking orientation to resist and/or prevent movement of the slide relative to the fulcrum.

18. A surgical system comprising:
a first implant support;
a second implant support;
a fulcrum including a first surface that defines a cavity for disposal of the first support and being rotatable relative to the fulcrum to fix the first surface with the first support at a selected position and a second surface being engageable with the second implant support; and
pliers engageable with the supports to compress and/or distract tissue.

19. A surgical system as recited in claim 18, wherein the second surface is movable relative to the fulcrum to engage the second support.

20. A surgical system as recited in claim 18, wherein the fulcrum includes a lock engageable with the second surface to fix the second surface in the locking orientation.
